(19) United States
(12) Patent Application Publication
(10) Pub. No.: US 2020/0345611 A1
(43) Pub. Date: Nov. 5, 2020

(54) COMPOSITION COMPRISING AT LEAST ONE CERAMIDE, AT LEAST ONE SPHINGOID BASE AND TRIETHYL CITRATE

(71) Applicant: Evonik Operations GmbH, Essen (DE)

(72) Inventors: Ursula Maczkiewitz, Essen (DE); Lisa Maus, Duisburg (DE); Claudia Dietl, Essen (DE); Monica Desiree van Logchem, Zevenbergen (NL); Maria Mecking, Bottrop (DE); Manuela Salmina-Petersen, Hamburg (DE); Heike Blasko-Begoihn, Essen (DE)

(73) Assignee: Evonik Operations GmbH, Essen (DE)

(21) Appl. No.: 16/826,599

(22) Filed: Mar. 23, 2020

(30) Foreign Application Priority Data
Apr. 30, 2019 (EP) 19171845.1

(51) Int. Cl. A61K 8/68; A61K 8/41; A61K 8/37

(52) U.S. Cl. CPC A61K 8/68; A61K 8/37; A61K 8/41

(57) ABSTRACT

The invention relates to a composition comprising at least one ceramide, at least one sphingoid base and triethyl citrate, a process for producing a formulation comprising at least one ceramide and the use of triethyl citrate for stabilization of a ceramide-containing formulation.

### CROSS REFERENCE TO RELATED APPLICATIONS

This application is a 35 U.S.C. § 119 patent application which claims the benefit of European Application No. 19171845.1 filed Apr. 30, 2019, which is incorporated herein by reference in its entirety.

### FIELD

The invention relates to a composition comprising at least one ceramide, at least one sphingoid base and triethyl citrate, a process for producing a formulation comprising at least one ceramide and at least one sphingoid base and to the use of triethyl citrate for stabilization of a ceramide-containing formulation.

### BACKGROUND

Ceramides are used in many cosmetic formulations. However, their low solubility and strong tendency to recrystallization complicate the stable incorporation into cosmetic formulations. Especially at temperatures just above freezing, formulations often suffer from inhomogeneities. In addition, microbial stabilization of ceramide-containing formulations is challenging since preservatives regularly adversely affect the physical stability of formulations.

The prior art addresses this problem by various delivery forms.

For instance, WO1998053797 describes encapsulated water-insoluble active ingredients having amphiphilic character, with a content of water and at least one surfactant from the group of esters of long-chain carboxylic acids with carboxylic acids comprising hydroxyl groups or salts thereof and esters of long-chain carboxylic acids with polyalcohols, in which ceramides may be the water-insoluble active ingredients.

WO1999029293 describes a composition for topical application, comprising a combination of a free sphingoid base and a ceramide.

JP2008297301 describes ceramides and phytosterol derivatives in triglyceride-containing oil solutions in liquid form for incorporation into dermatological preparations.

### SUMMARY

The object of the invention was to provide compositions comprising ceramides which have excellent microbial and physical stability.

### DETAILED DESCRIPTION

It has been found that, surprisingly, the compositions described below are able to solve the problem addressed by the invention.

The present invention therefore relates to compositions comprising A) at least one ceramide, B) at least one sphingoid base and C) triethyl citrate.

The invention further relates to a process for producing a formulation comprising at least one ceramide and the use of triethyl citrate for stabilization of ceramide-containing formulation.

An advantage of the present invention is that the compositions according to the invention have increased storage stability and are thus slower to change in their nature over time, particularly with regard to their viscosity, compared to ceramide-containing compositions according to the prior art.

Another advantage of the present invention is that the compositions according to the invention tolerate a higher number of freeze-thaw steps without significant loss of viscosity, compared to ceramide-containing compositions according to the prior art.

One advantage of the composition according to the invention is that the composition in formulations has superior sensory properties which lead to an improved skin feel and/or hair feel.

It is a further advantage of the composition according to the invention that the composition in formulations stimulates in vitro human follicular dermal papilla cells (HFDPCs) to proliferate, which in vivo equates to stimulation of hair growth.

It is a further advantage of the composition according to the invention that the composition in formulations has improved distributability compared to the individual components.

It is a further advantage of the composition according to the invention that the composition in formulations has improved absorption compared to the individual components.

It is a further advantage of the composition according to the invention that the composition in formulations has reduced oiliness compared to the individual components.

It is a further advantage of the composition according to the invention that the composition in formulations has reduced waxiness compared to the individual components.

It is a further advantage of the composition according to the invention that the composition in formulations has improved glidability compared to the individual components.

It is a further advantage of the composition according to the invention that the composition in formulations has reduced tackiness compared to the individual components.

It is a further advantage of the composition according to the invention that the composition in formulations has improved silkiness/velvetiness compared to the individual components.

In the context of the present invention, the term “ceramide” is understood to mean acylated sphingoid bases, where the sphingoid bases are preferably selected from sphingosine, sphinganine, 6-hydroxysphingosine and phytosphingosine.

The “pH” in connection with the present invention is defined as the value which is measured for the relevant composition at 25° C. after stirring for five minutes using a pH electrode calibrated in accordance with ISO 4319 (1977).

Unless stated otherwise, all percentages (%) given are percentages by mass.

Preferred compositions according to the invention are characterized in that the ceramide is selected from the group ceramide NP, ceramide AP, ceramide EOP, ceramide NDS, ceramide ADS, ceramide EODS, ceramide NS, ceramide AS, ceramide EOS, ceramide NH, ceramide AH and ceramide EOH.

It is preferred in accordance with the invention that the composition according to the invention comprises a sphingoid base selected from the group sphingosine, sphinganine, 6-hydroxysphingosine and phytosphingosine.

A particularly preferred composition according to the invention is characterized in that the ceramide is selected from the group comprising ceramide NP and ceramide AP, and the sphingoid base is selected from phytosphingosine.

In particular, it is preferred that the composition according to the invention comprises ceramide NP and ceramide AP as ceramide, and the sphingoid base is selected from phytosphingosine.

In this preferred embodiment of the composition according to the invention, it is preferred that ceramide NP and ceramide AP is present in a ratio by weight of 1.5 to 2.5:1 in the composition according to the invention.

Preferred compositions according to the invention are characterized in that the ratio by weight of components A) to B) to C) is from 2 to 6:1 to 2:1 to 5, preferably 2 to 6:1 to 2:1 to 2, wherein the weight of the respective component refers to all ceramides, sphingoid bases and triethyl citrate in the composition.

Preferred compositions according to the invention are active ingredient concentrates having a high active content; such a preferred composition according to the invention is characterized in that components A) and B) are present in total in an amount of 1.0% by weight to 9.0% by weight, preferably 1.5% by weight to 5.0% by weight, particularly preferably 2.0% by weight to 3.0% by weight, wherein the percentages by weight refer to the total composition.

From these active ingredient concentrates, formulations, especially cosmetic formulations, can be produced by mixing, in which the active ingredient concentration is reduced compared to the aforementioned concentrates; such an alternative, preferred composition according to the invention is characterized in that components A) and B) are present in total in an amount of 0.02% by weight to 0.60% by weight, preferably 0.03% by weight to 0.40% by weight, particularly preferably 0.05% by weight to 0.30% by weight, wherein the percentages by weight refer to the total composition.

Such alternative preferred compositions according to the invention are particularly formulations, particularly preferably cosmetic formulations.

The formulations according to the invention can further comprise at least one additional component selected from the group of

emollients,
emulsifiers,
thickeners/viscosity regulators/stabilizers,
UV light protection filters,
antioxidants,
hydrotropes (or polyols),
solids and fillers,
film formers,
pearlescence additives,
deodorant and antiperspirant active ingredients,
insect repellents,
self-tanning agents,
preservatives,
conditioning agents,
perfumes,
dyes,
odor absorbers,
cosmetic active ingredients,
care additives,
superfatting agents,
solvents.

Substances which can be used as exemplary representatives of the individual groups are known to those skilled in the art and can be found for example in German application DE 102008001788.4. This patent application is hereby incorporated as reference and thus forms part of the disclosure.

As regards further optional components and the amounts used of these components, reference is made expressly to the relevant handbooks known to those skilled in the art, for example K. Schrader, “Grundlagen and Rezepturen der Kosmetika [Cosmetics—fundamentals and formulations]”, 2nd edition, pages 329 to 341, Hilthig Buch Verlag Heidelberg.

The amounts of the particular additives are determined by the intended use.

Typical boundary formulations for the respective applications are known prior art and are contained for example in the brochures of the manufacturers of the particular base and active ingredients. These existing formulations can generally be adopted unchanged. However, if required, for adjustment and optimization, the desired modifications can be undertaken by simple tests without complication.

It is preferred in accordance with the invention that the inventive composition of the present invention comprises D) cholesterol, in particular as active ingredient concentrates according to the invention, preferably in an amount of 0.1% by weight to 3.0% by weight, preferably 0.3% by weight to 2.0% by weight, particularly preferably 0.4% to 1.0% by weight, wherein the percentages by weight refer to the total composition.

Compositions of the present invention which are preferred according to the invention are emulsions, in particular oil-in-water emulsions.

Therefore, it is preferred in accordance with the invention that the inventive composition of the present invention comprises E) at least one emulsifier, in particular as active ingredient concentrates according to the invention in an amount of 0.1% by weight to 3.0% by weight, preferably 0.3% by weight to 2.0% by weight, particularly preferably 0.4% to 1.0% by weight, wherein the percentages by weight refer to the total composition.

The term “emulsifier” in the context of the present invention is understood to mean organic substances having surface-active properties which may have the ability to lower the surface tension of water at 20° C. and at a concentration of 0.5% by weight, based on the total composition, to less than 45 mN/m, and for which there is no pH between 2 and 12, in which at 20° C., at least 50 mol % of the molecules are ionically charged. Thus, these substances are overall mostly uncharged from the outside at all pH values between 2 and 12.

The surface tension is determined here by the ring method in accordance with du Noüy at 20° C. Emulsifiers preferably present are selected from the group of acyl lactylates (especially sodium lauroyl lactylate), ethoxylated fatty alcohols (especially ceteareth-25), ethoxylated fatty acids (especially PEG_100 stearates), polyglycerol esters (especially polyglyceryl-3 methylglucose distearate, polyglyceryl-6 stearate, polyglyceryl-6 behenate), glycerol esters, (especially glyceryl stearate citrate), ethoxylated sorbitan esters and lecithin.

The term “acyl lactylate” in the context of the present invention is understood to mean salt-form reaction products of fatty acid with lactic acid and/or polylactic acid.

A particularly preferred composition according to the invention is characterized in that said composition has a pH in the range of 4.0 to 8.0, preferably 4.5 to 7.4, particularly preferably 5.5 to 6.9, alternatively particularly preferably 4.0 to 5.4.

The present invention further relates to a process for producing a formulation comprising A) at least one ceramide, comprising the process steps of:

a) providing the at least one ceramide and providing C) triethyl citrate;
b) mixing components A) and C), together with water to give a solution, in particular a complete solution, of component A).

The process according to the invention is preferably one for producing a formulation comprising

A) at least one ceramide and B) at least one sphingoid base, comprising the process steps of:
a) providing the at least one ceramide and providing C) triethyl citrate and providing the at least one sphingoid base;
b) mixing components A) and C) and B) together with water, to give a solution of component A).

The formulations prepared by the process according to the invention are preferably the compositions according to the invention described above, wherein preferred compositions according to the invention are likewise preferably prepared by the process according to the invention.

Thus, for example, in the process according to the invention the ratio by weight of components A) to B) to C) is preferably used in a ratio by weight of from 2 to 6:1 to 2:1 to 5, preferably 2 to 6:1 to 2:1 to 2, wherein the weight of the respective component refers to all ceramides, sphingoid bases and triethyl citrate used in the process.

The formulations prepared by the process according to the invention are in particular physically stable formulations. In the context of the present invention, the term “physically stable formulations containing at least one ceramide” is understood in particular to mean formulations which in particular do not exhibit any crystallization of the ceramides and the sphingoid base and no separation or inhomogeneity after six months of storage at 25° C.

It is advantageous in accordance with the invention and therefore preferred when the process according to the invention is characterized in that process step b) is carried out at least partially in a temperature range of 70° C. to 110° C., preferably 75° C. to 100° C., particularly preferably 80° C. to 90° C.

It is particularly preferred in accordance with the invention when the process according to the invention is characterized in that in process step b), component A), preferably and B), are mixed with water to give a homogeneous solution in a temperature range of 70° C. to 110° C., preferably 75° C. to 100° C., particularly preferably 80° C. to 90° C., and component C) is mixed with the homogeneous solution in a temperature range of 5° C. to 50° C., preferably 10° C. to 40° C., particularly preferably 15° C. to 30° C.

A preferred process according to the invention is characterized in that in process step b) the solution has a pH range of 4.0 to 8.0, preferably 4.5 to 7.4, particularly preferably 5.5 to 6.9, alternatively particularly preferably of 4.0 to 5.4.

A particularly preferred process according to the invention is characterized in that in process step b) at least one emulsifier is present.

In accordance with the invention, this is preferably mixed with the other components in process step b) in a temperature range of 70° C. to 110° C., preferably 75° C. to 100° C., particularly preferably 80° C. to 90° C.

Emulsifiers preferably used are selected from the group consisting of sodium lauroyl lactylate, ethoxylated fatty alcohols (especially ceteareth-25), ethoxylated fatty acids (especially PEG 100 stearates), polyglycerol esters (especially polyglyceryl-3 methylglucose distearate, polyglyceryl-6 stearate, polyglyceryl-6 behenate), glycerol esters, (especially glyceryl stearate citrate), ethoxylated sorbitan esters and lecithin.

The preferred ratio by weight of components A) and B) in total to the emulsifier in method step b) is from 1:1 to 1:20, preferably from 1:2 to 1:10, wherein the weights refer to all ceramides, sphingoid bases and emulsifiers present.

The further preferred ratios by weight of the individual components to one another in the process according to the invention correspond to the preferred compositions according to the invention. The same applies to the preferred components A), B) and C) and emulsifier.

It has been found, quite surprisingly, that triethyl citrate has an advantageous effect on the stabilization of ceramide-containing formulations; therefore, the present invention further relates to the use of triethyl citrate for physical stabilization of a ceramide-containing formulation, in particular with regard to homogeneity, and/or for preventing crystallization of at least one ceramide in a formulation, and/or for microbial stabilization of a ceramide-containing formulation, in particular with respect to at least one selected from S. aureaus, P. aeruginosa, E. coli, C. albicans and A. brasiliensis.

The examples adduced hereinafter illustrate the present invention by way of example, without any intention of restricting the invention, the scope of application of which is apparent from the entirety of the description and the claims, to the embodiments specified in the examples.

### EXAMPLES

### Example 1: Microbial Stability

Ceramide-containing formulations were prepared using various preservative combinations. The composition of the ceramide-containing formulation is given in Table 1 (the concentration data are % by weight):

TABLE 1
Ceramide-containing formulations

| Phase | Ingredient | w/w % |
|---|---|---|
| A | Sodium Lauroyl Lactylate | 10.0 |
| | Ceramide NP | 1.12 |
| | Ceramide AP | 0.53 |
| | Phytosphingosine | 0.54 |
| | Cholesterol | 0.50 |
| B | Water | to 100.0 |
| | Carbomer | 0.30 |
| | Sodium hydroxide (10% in water) | to pH 6 |
| C | Xanthan Gum | 0.30 |
| D | Preservative | x |

The production was as follows:

Phase A and B were heated to 85° C. Then, phase A was added slowly to phase B with stirring using an Ystrahl stirrer. The mixture was then cooled to 70° C., phase C was added and homogenized with the Ystrahl until the formulation was homogeneous. The mixture was then cooled to 25° C. with stirring.

To this formulation, as a last step, the various preservatives were added and stirred homogeneously.

The formulations thus obtained were subjected to a preservation stress test (PST). This test was carried out according to the corresponding procedure from the European Pharmacopoeia 7.0, 5.1.3 Efficacy of antimicrobial preservation. If the test did not fulfill at least criterion B for all organisms, then the PST was considered as “failed”.

The microorganisms tested were S. aureaus, P. aeruginosa, E. coli, C. albicans and A. brasiliensis:

The following table summarizes the results.

TABLE 2
Results of the preservation stress test

| | | | CFU |
|---|---|---|---|
| x % Preservative | Inventive | Comprises | result |
| 1% Euxyl PE 9010 + | No | Phenoxyethanol, | failed |
| 0.2% Sensiva SC 50 | | Ethylhexylglycerin | |
| 1.5% Sensiva PA 40 | No | Phenylpropanol, | failed |
| | | Propanediol, Caprylyl | |
| | | Glycol, Tocopherol | |
| 2% Sensiva SC 10 | No | Caprylyl Glycol, | failed |
| | | Ethylhexylglycerin | |
| 1% Euxyl K 900 | No | Benzyl alcohol, | failed |
| | | Ethylhexylglycerin, | |
| | | Tocopherol | |
| 3% dermosoft OMP | No | Methylpropanediol, | failed |
| | | Caprylyl Glycol, | |
| | | Phenylpropanol | |
| 1.5% Verstatil SL + 3% | No | Sodium Levulinate, | failed |
| dermosoft OM | | Potassium Sorbate, | |
| | | Methylpropanediol, | |
| | | Caprylyl Glycol | |
| 3% dermosoft OMP + 2% | Yes | Methylpropanediol, | passed |
| dermofeel tec eco | | Caprylyl Glycol, | |
| | | Phenylpropanol, | |
| | | Triethyl citrate | |
| 2% Verstatil SL + 2% | Yes | Sodium Levulinate, | passed |
| dermofeel tec eco | | Potassium Sorbate, | |
| | | Triethyl citrate | |
| 1.5% Verstatil TBO | Yes | Triethyl citrate, | passed |
| | | Caprylyl | |
| | | Glycol, Benzoic | |
| | | Acid | |

Surprisingly, it has been found that preservative combinations comprising triethyl citrate meet the requirements of the preservation stress test, whereas preservative combinations without triethyl citrate fail to do so.

### Example 2: Physical Stability

The ceramide-containing formulations from Example 1 with the various preservatives were additionally tested for recrystallization of the ceramides. This was achieved using an Olympus IX83 light microscope. Table 3 summarizes the results of the microscope examination.

TABLE 3
Results of microscope examinations

| | | | Result of the |
|---|---|---|---|
| | | | microscope |
| Preservative | Inventive | Composition | examination |
| 1% Euxyl PE | No | Phenoxyethanol, | Recrystallization |
| 9010 + | | Ethylhexylglycerin | of ceramides |
| 0.2% Sensiva | | | |
| SC 50 | | | |
| 1.5% Sensiva | No | Phenylpropanol, | Recrystallization |
| PA 40 | | Propanediol, | of ceramides |
| | | Caprylyl Glycol, | |
| | | Tocopherol | |
| 2% Sensiva SC 10 | No | Caprylyl Glycol, | Recrystallization |
| | | Ethylhexylglycerin | of ceramides |
| 1% Euxyl K 900 | No | Benzyl alcohol, | Recrystallization |
| | | Ethylhexylglycerin, | of ceramides |
| | | Tocopherol | |
| 3% dermosoft OMP + | Yes | Methylpropanediol, | No recrystallization |
| 2% dermofeel | | Caprylyl | |
| tec eco | | Glycol, | |
| | | Phenylpropanol, | |
| | | Triethyl citrate | |
| 2% Verstatil SL + | Yes | Sodium Levulinate, | No recrystallization |
| 2% dermofeel tec | | Potassium | |
| eco | | Sorbate, Triethyl | |
| | | citrate | |
| 1.5% Verstatil TBO | Yes | Triethyl citrate, | No recrystallization |
| | | Caprylyl Glycol, | |
| | | Benzoic Acid | |

Surprisingly, it has also been found here that triethyl citrate prevents the recrystallization of ceramides, whereas preservatives which do not comprise triethyl citrate were unable to do so. In the absence of triethyl citrate, the ceramide-containing formulations showed a clear recrystallization of the ceramides.

1. A composition comprising
A) a ceramide,
B) a sphingoid base and
C) triethyl citrate.

2. The composition according to claim 1, wherein
the ceramide is selected from the group consisting of ceramide NP, ceramide AP, ceramide EOP, ceramide NDS, ceramide ADS, ceramide EODS, ceramide NS, ceramide AS, ceramide EOS, ceramide NH, ceramide AH and ceramide EOH.

3. The composition according to claim 1, wherein
the sphingoid base is selected from the group consisting of sphingosine, sphinganine, 6-hydroxysphingosine and phytosphingosine.

4. The composition according to claim 1, wherein
the ceramide is selected from the group consisting of ceramide NP and ceramide AP, and
the sphingoid base is selected from phytosphingosine.

5. The composition according to at claim 1, wherein
the ceramide present is ceramide NP and ceramide AP, and
the sphingoid base is selected from phytosphingosine.

6. The composition according to claim 1, wherein the ratio by weight of components A) to B) to C) is from 2 to 6:1 to 2:1 to 5.

7. The composition according to claim 1, wherein
ceramide NP and ceramide AP is present in a ratio by weight from 1.5 to 2.5:1.

8. The composition according to claim 1, wherein components A) and B) are present in total in an amount of 1.0% by weight to 9.0% by weight, wherein the percentages by weight refer to the total composition.

9. The composition according to claim 1, wherein components A) and B) are present in total in an amount of 0.02% by weight to 0.60% by weight, wherein the percentages by weight refer to the total composition.

10. The composition according to claim 1, wherein said composition comprises
D) cholesterol, in an amount of 0.1% by weight to 3.0% by weight, wherein the percentages by weight refer to the total composition.

11. The composition according to claim 1, wherein said composition has a pH in the range of 4.0 to 8.0.

12. A process for producing a formulation comprising A) at least one ceramide, comprising the process steps of:
a) providing the at least one ceramide and providing C) triethyl citrate;
b) mixing components A) and C) together with water, to give a solution of component A).

13. The process according to claim 12, wherein process step b) is carried out at least partially in a temperature range of 70° C. to 110° C.

14. The process according to claim 12, wherein in process step b) the solution has a pH range of 4.0 to 8.0.

15. (canceled)

16. The composition according to claim 1, wherein components A) and B) are present in total in an amount of 1.5% by weight to 5.0% by weight, wherein the percentages by weight refer to the total composition.

17. The composition according to claim 1, wherein components A) and B) are present in total in an amount of 0.05% by weight to 0.30% by weight, wherein the percentages by weight refer to the total composition.

18. The composition according to claim 1, wherein said composition comprises
D) cholesterol, in an amount of 0.4% by weight to 1.0% by weight, wherein the percentages by weight refer to the total composition.

19. The composition according to claim 1, wherein said composition has a pH in the range of 4.0 to 5.4.

20. The process according to claim 12, wherein process step b) is carried out at least partially in a temperature range of 80° C. to 90° C.

21. The process according to claim 12, wherein in process step b) the solution has a pH range of 4.5 to 7.4.
